(19) United States
(12) Patent Application Publication
(10) Pub. No.: US 2025/0255515 A1
(43) Pub. Date: Aug. 14, 2025

(54) System, Method, and Computer Program Product for Tracking Cognitive Status Based On Repeated Mobility Assessments

(71) Applicant: Celloscope Ltd., Tel Aviv (IL)

(72) Inventors: Yuval NAVEH, Haifa (IL); Tamir ALOUSH, Kiryat Ono (IL); Levi TEITZ, Tel Aviv (IL); Shahar DAVIDSON, Rehovot (IL); Tomer SHUSSMAN, Kfar Saba (IL)

(21) Appl. No.: 19/051,712

(22) Filed: Feb. 12, 2025

Related U.S. Application Data
(63) Continuation-in-part of application No. 18/939,288, filed on Nov. 6, 2024.
(63) Continuation-in-part of application No. 18/975,890, filed on Dec. 10, 2024.
(63) Continuation-in-part of application No. 19/049,400, filed on Feb. 10, 2025.
(60) Provisional application No. 63/596,479, filed on Nov. 6, 2023.
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,757, filed on Feb. 26, 2024.
(60) Provisional application No. 63/557,762, filed on Feb. 26, 2024.

(51) Int. Cl. A61B 5/11; A61B 5/00; G16H 50/70

(52) U.S. Cl. CPC A61B 5/112; A61B 5/1126; A61B 5/4088; A61B 5/6898; A61B 5/7264; A61B 5/746; G16H 50/70; A61B 2560/0462; A61B 2562/0219

(57) ABSTRACT

A health monitoring method, system and computer program product comprising using a cellphone having an IMU, to (typically repeatedly) measure at least one gait parameter of a human bearing the cellphone during a first period in which the human is known to be dual-tasking; and/or at least one gait parameter of the human bearing the cellphone during a second period in which the human is known not to be dual-tasking; and/or using a hardware processor to receive the gait parameters and/or to compute at least one difference (in value e.g.) between gait parameter/s during the first period and (typically the same) gait parameter/s during the second periods, and, accordingly, to generate a cognitive health alert at least once e.g. according to an alert generation criterion defined over at least the difference/s so computed.

### CROSS-REFERENCE TO RELATED APPLICATIONS

The present application is a continuation-in-part of application Ser. No. 18/939,288, filed Nov. 6, 2024, and of application Ser. No. 18/975,890, filed Dec. 10, 2024, and of application Ser. No. 19/049,400, filed Feb. 10, 2025, the entire contents of each of which being hereby fully incorporated herein by reference. The present application further claims benefit, directly or indirectly, of the following provisional applications, the entire contents of each of which being fully incorporated herein by reference: Application No. 63/557,740, filed Feb. 26, 2024; Application No. 63/557,747, filed Feb. 26, 2024; Application No. 63/557,753, filed Feb. 26, 2024 and entitled “Mobility Assessment in Patients with Cognitive Decline”; Application No. 63/557,757, filed Feb. 26, 2024, Application No. 63/557,762, filed Feb. 26, 2024; Application No. 63/596,479, filed Nov. 6, 2023; and Application No. 63/612,587, filed Dec. 20, 2023.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

Voice activity detection and speaker recognition are known.

According to this online reference www.alz.org/professionals/health-systems-medical-professionals/clinical-resources/cognitive-assessment-tools, “cognitive assessment tools are used to identify individuals who may need additional evaluation. No one tool is recognized as the best brief assessment to determine if a full dementia evaluation is needed. However, the expert workgroup identified several instruments suited for use in primary care based on the following: administration time 55 minutes, validation in a primary care or community setting, psychometric equivalence or superiority to the Mini-Mental State Exam (MMSE), easy administration by non-physician staff and relatively free of educational, language and/or cultural bias. For a definitive diagnosis of mild cognitive impairment or dementia, individuals who fail any of these tests should be evaluated further or referred to a specialist”.

The Impact of Mild Cognitive Impairment on Gait and Balance: A Systematic Review and Meta-Analysis of Studies Using Instrumented Assessment (Bahureksa et al. 2017) pubmed.ncbi.nlm.nih.gov/27172932/discusses the impact of mild cognitive impairment on gait and balance.

Dual-Task Gait as a Predictive Tool for Cognitive Impairment in Older Adults: A Systematic Review (Ramirez and Gutierrez 2021) pubmed.ncbi.nlm.nih.gov/35002676/ discusses dual-task gait as a predictive tool for cognitive impairment in older adults.

A Feb. 27, 2020 article describes OneStep as an artificial intelligence-powered mobile software platform which provides ongoing health diagnostics, offers continuous progress feedback and direct connection to a therapist, and continuously detects and analyzes gait through a smartphone's sensors, whether it is in the end-user's pocket, hand, or backpack. The platform discerns the end-user's gait parameters such as stride length, hip range, and step rate. www.jpost.com/opinion/hillels-tech-corner-tel-aviv-start-up-taking-tech-one-step-further-619140

Other studies in the field include:

Byun, Seonjeong, Ji Won Han, Tae Hui Kim, Kayoung Kim, Tae Hyun Kim, Jae Young Park, Seung Wan Suh, et al. 2018. “Gait Variability Can Predict the Risk of Cognitive Decline in Cognitively Normal Older People.” Dementia and Geriatric Cognitive Disorders 45 (5-6): 251-61.

Dimitriadis, Stavros I., Nikolaos A. Laskaris, Malamati P. Bitzidou, loannis Tarnanas, and Magda N. Tsolaki. 2015. “A Novel Biomarker of Amnestic MCI Based on Dynamic Cross-Frequency Coupling Patterns during Cognitive Brain Responses.” Frontiers in Neuroscience 9 (October): 350.

Montero-Odasso, Manuel M., Yanina Sarquis-Adamson, Mark Speechley, Michael J. Borrie, Vladimir C. Hachinski, Jennie Wells, Patricia M. Riccio, et al. 2017. “Association of Dual-Task Gait With Incident Dementia in Mild Cognitive Impairment: Results From the Gait and Brain Study.” JAMA Neurology 74 (7): 857-65.

Muir, Susan W., Karen Gopaul, and Manuel M. Montero Odasso. 2012. “The Role of Cognitive Impairment in Fall Risk among Older Adults: A Systematic Review and Meta-Analysis.” Age and Ageing 41 (3): 299-308.

Rosso, Andrea L., Andrea L. Metti, Kimberly Faulkner, Mark Redfern, Kristine Yaffe, Lenore Launer, C. Elizabeth Shaaban, Neelesh K. Nadkarni, and Caterina Rosano. 2019. “Complex Walking Tasks and Risk for Cognitive Decline in High Functioning Older Adults.” Journal of Alzheimer's Disease: JAD 71 (s1): S65-73.

Taniguchi, Yu, Hiroto Yoshida, Yoshinori Fujiwara, Yutaka Motohashi, and Shoji Shinkai. 2012. “A Prospective Study of Gait Performance and Subsequent Cognitive Decline in a General Population of Older Japanese.” The Journals of Gerontology. Series A, Biological Sciences and Medical Sciences 67 (7): 796-803.

OneStep is an FDA-listed medical app, downloadable from GooglePlay, that uses smartphone motion sensors to provide immediate, clinically-validated feedback on gait inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### Summary of Certain Embodiments

Certain embodiments of the present invention seek to provide circuitry typically comprising at least one processor in communication with at least one memory, with instructions stored in such memory executed by the processor to provide functionalities which are described herein in detail.

Any functionality described herein may be firmware-implemented or processor-implemented, as appropriate.

Attention needs to be directed to the frequency at which cognitive assessments are performed, if at all. Even in the most skilled nursing facilities and high-end senior living communities, which constitute a small minority of the care settings available to the population as a whole, resources to run such assessments, every week, and for everyone, are lacking. Also, many seniors and others at risk for cognitive decline are not evaluated even once a year, again seemingly due to lack of resources. Some at-risk persons are not evaluated at all. There are no universally accepted recommendations, preferably based on cost-benefit analysis, for an ideal time-interval between cognitive assessments.

Certain embodiments seek to provide continuous assessment of cognitive status typically using repeated and automatic, e.g. continuous gait parameter measurement, e.g. by measurement of passive gait.

According to one embodiment, mobility assessment, e.g. gait parameter measurement and/or thresholding thereof, are “continuous” in the sense that t seconds are required to compare measurements from a known dual processing interval to measurements from a known non-dual (aka single) processing interval, and then immediately when t finishes, the system repeats the process. The intervals may each be a window of a few seconds in length, and the intervals used in repetition n may overlap with the intervals used in repetition n+1.

It is appreciated that many more measurements over time are collected (compared to in-clinic assessments), including remote administered mobility tests (including dual tasking), passive gait, and in-clinic/controlled/supervised measurements.

Certain embodiments seek to provide all or any subset of: monitoring mobility e.g. gait repeatedly, establishing baselines, repeatedly comparing the current gait/mobility status and the baselines, and performing dual-task protocol assessment. A given user's baselines may be established, say, on registration, e.g. as part of onboarding the system, and then occasionally or periodically, e.g. every month afterwards.

It is appreciated that a given user may have plural baselines stored in the system, e.g. her onboarding baseline, her baseline before and after clinical events such as injuries or interventions (e.g., surgeries), and ongoing baselines (e.g., “a month ago”).

A baseline typically comprises measurements' values at a specific time. For example, the system may store an onboarding/registration baseline of gait speed/dual-tasking/any other measurements. Alternatively, or in addition, if plural measurements are made each week, the resulting values may be averaged, and the central tendency, e.g. average (and typically also the standard deviation) may be stored and may be used as a baseline to which new values are compared, resulting in a difference, which may then be used as a threshold.

Certain embodiments comprise a mobile device with IMU, a mobile app, a caregiver dashboard, and a processor configured to receive data and communicate with the mobile app and the caregiver dashboard. Typically, the processor continuously assesses the mobile app's user functional status and enables the caregiver to implement different care actions based on at least one mobility measure. Any suitable care actions and/or mobility measures may be employed, e.g. as described herein.

Certain embodiments seek to provide mobility assessment in patients with cognitive decline.

At least the following embodiments are included in the scope of the present invention:

Embodiment 1. A health monitoring method comprising: using a cellphone having an IMU, to measure: at least one gait parameter of a human bearing the cellphone during a first period in which the human is known to be dual-tasking; and/or at least one gait parameter of the human bearing the cellphone during a second period in which the human is known not to be dual-tasking; and/or using a hardware processor to receive the gait parameters and to compute at least one difference between the gait parameters during the first and second periods, and, accordingly, to generate a cognitive health alert.

Embodiment 2. A method according to any of the preceding embodiments and wherein the health alert comprises a cognitive status indication provided by automatically and repeatedly, e.g. according to a programmed schedule or periodically, thresholding the at least one difference, to provide close tracking of cognitive status which results from repeatedly receiving up-to-date gait parameters and repeatedly using the hardware processor to compute the difference.

It is appreciated that greater frequency of monitoring is achieved using this paradigm, which is workable even in a non-clinic setting, relative to the sparse frequency of data points captured in clinic scenarios, even if the above thresholding is performed only once a week.

Embodiment 3. A method according to any of the preceding embodiments and wherein the cognitive status indication comprises an output indication sensible by humans which may be generated at a location remote from the cellphone.

Embodiment 4. A method according to any of the preceding embodiments and wherein the cognitive status indication comprises an output signal fed to an external computer system, which receives and conducts comparisons between the output signal and between similar output signals from a multiplicity of persons bearing cellphones and, responsively, continuously re-ranks persons to prioritize persons according to the output signals provided by the method and by the persons' cellphones.

Embodiment 5. A method according to any of the preceding embodiments and wherein the output indication is generated each time the difference falls below at least one baseline range of gait parameter values.

Embodiment 6. A method according to any of the preceding embodiments and wherein the hardware processor used to compute the at least one difference, comprises the cellphone's hardware processor.

Embodiment 7. A method according to any of the preceding embodiments and wherein at least one of the first and second periods is determined automatically by determining whether a human is or is not dual-tasking during at least one time-period, e.g. constantly, to enable cognitive monitoring, even without any active cooperation from the user.

Embodiment 8. A method according to any of the preceding embodiments and wherein a single cognitive status indication is used to generate plural output signals fed to plural external computer systems respectively, each of which is tasked with multifactorial data processing tasks respectively, thereby to define plural multifactorial data processing tasks, all of which depend inter alia on cognitive status.

Embodiment 9. A health monitoring system comprising: a hardware processor interfacing with a cellphone's IMU, e.g. to collect: at least one gait parameter of a human bearing the cellphone during a first period in which the human is known to be dual-tasking; and/or at least one gait parameter of the human bearing the cellphone during a second period in which the human is known not to be dual-tasking; and wherein the hardware processor may be configured for computing at least one difference between the gait parameters during the first and second periods, and, accordingly, commanding the cellphone to generate a cognitive health alert which is typically sensible by a caregiver.

Embodiment 10. A method according to any of the preceding embodiments and wherein the gait parameters are measured passively without user cooperation, thereby to enable the cognitive health alert to be generated without enlisting user cooperation.

Embodiment 11. A method according to any of the preceding embodiments wherein the first and second periods are selected to be comparable.

Embodiment 12. A computer program product, comprising a non-transitory tangible computer readable medium having computer readable program code embodied therein, the computer readable program code adapted to be executed to implement a health monitoring method comprising: using a cellphone having an IMU, e.g. to measure: at least one gait parameter of a human bearing the cellphone during a first period in which the human is known to be dual-tasking; and/or at least one gait parameter of the human bearing the cellphone during a second period in which the human is known not to be dual-tasking; and/or using a hardware processor to receive the gait parameters and to compute at least one difference between the gait parameters during the first and second periods, and, accordingly, to generate a cognitive health alert.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer-readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” may be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

Certain embodiments of the present invention are illustrated in the following drawings; in the block diagrams, arrows between modules may be implemented as APIs, and any suitable technology may be used for interconnecting functional components or modules illustrated herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol.

FIGS. 1a and 1b are example methods for determining, using passive walks, that the user executes, without having been prompted and without cooperation, a level of a given user's cognitive cost aka cognitive load for dual-tasking.

FIG. 2 is an example method for extraction of certain environmental conditions.

FIG. 3 is a table useful in understanding certain embodiments.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional, or logical components described and illustrated herein may be implemented in various forms, for example as hardware circuits, such as but not limited to custom VLSI circuits or gate arrays or programmable hardware devices such as but not limited to FPGAs, or as software program code stored on at least one tangible or intangible computer-readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa.

Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may, in fact, include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data may be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

A health monitoring system, method, and computer program product are provided herein, in which a cellphone having an accelerometer may be used to measure at least one gait parameter of a human bearing the cellphone during a first period in which the human is known to be dual-tasking; and at least one gait parameter of the human bearing the cellphone during a second period in which the human is known not to be dual-tasking. Also, a hardware processor may be used to compute difference/s between the gait parameters during the first and second periods, and, accordingly, to generate health alert/s typically comprising an output indication which may be sensible by humans and/or may be fed to an external computerized system. The health alert may include a cognitive status indication provided by thresholding continuous tracking of cognitive status which results from continuously using the hardware processor to compute the difference.

Any suitable gait parameters may be employed for this purpose, such as, say, gait speed and/or stride time and/or stride length and/or step length. Alternatively or in addition, and by way of non-limiting example, cadence, heel strike (a point in a user's gait cycle where the user's heel first contacts the ground, toe off (a point in the user's gait cycle where the user's toes push off from the ground), stance asymmetry, step length asymmetry, and any spatio-temporal measure of gait may be used as gait parameters; other possible parameters include variabilities in any such parameters, e.g. gait speed variability, which may be computed as the covariance of the gait speed between consecutive steps.

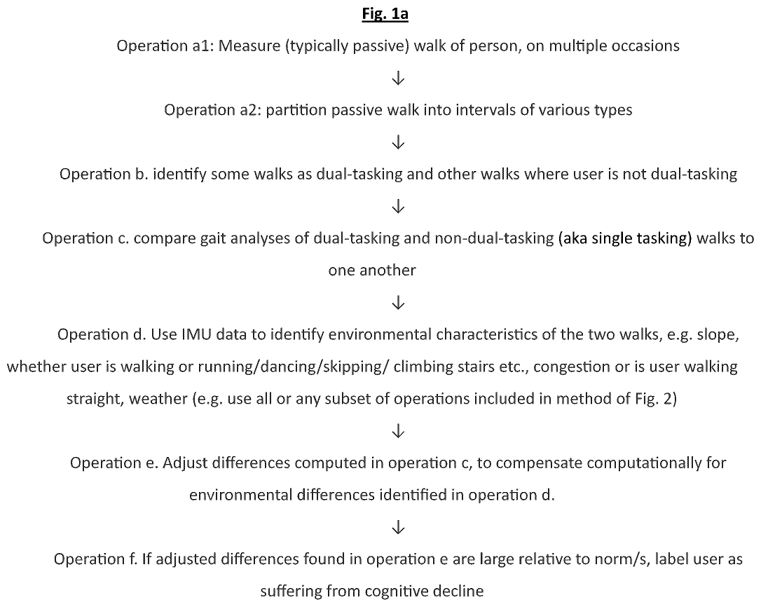

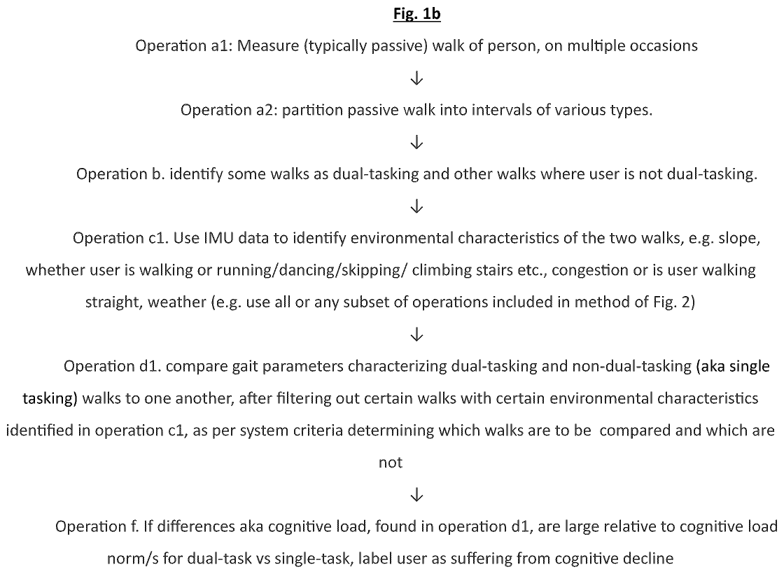

FIGS. 1a-1b are simplified flowchart illustrations of an example method for determining, using passive walks, that the user executes, without having been prompted and without cooperation, a level of a given user's cognitive cost for dual-tasking. The method of FIG. 1a may include all or any subset of the following operations, suitably ordered e.g. as follows:

Operation a1: Measure (typically passive) walk of person, on multiple occasions

Operation a2: partition passive walk into intervals of various types.

It is appreciated that co-owned U.S. patent application Ser. No. 18/975,890 “Advanced Pedestrian Navigation Based on Inertial Gait Analysis and GPS Data”, incorporated by reference herein in its entirety, of which this application is a continuation-in-part, describes inter alia how to partition motion, measured using an IMU (inertial Measurement Unit), into intervals characterized or classified using types such as, but not limited to, “gait” aka “walk intervals”, say, as opposed to, say, “position change” aka “device transition” intervals which may be indicative of dual-tasking (e.g. if the user is performing a mobility action during the interval, which may be detected e.g. as described below with reference to 50vii) given that the phone changes its bodily position (e.g. from user's pocket to her hand) rapidly and/or extensively in such intervals According to certain embodiments, “position change” intervals are compared to walks which may not include any dual processing.

Operation b. Discern whether the user is dual-tasking on each occasion, e.g. by using IMU data of the user's communication device's IMU, to identify whether the user is engaging in non-mobility related activity as he walks, including identifying some walks as dual-tasking walks, and other walks as walks in which the user is not dual-tasking.

Operation c. Computing difference/s between at least one measured walk which the system identified as a dual-tasking walk, with at least one measured walk (aka “regular walk”) which the system has identified as not being a dual-tasking walk—the system may be configured to compare gait analyses of these two walks to one another. The gait analysis may provide values for each of a set of gait mobility measures aka gait parameters, each typically averaged over the walk: spatiotemporal (e.g., all or any subset of gait speed, cadence, stride length, double support, single support). The system may then compute the difference, for each such average, between the regular walk and the dual task walk values, and may divide the difference by the regular walk average to present a percentage, aka dual task or cognitive cost, for that mobility measure. For example, for a given person, the cognitive cost of dual-tasking may be 23% for gait speed, and 19% for cadence.

Operation d. Use IMU data to identify environmental/contextual characteristics of the two walks, e.g. as described elsewhere herein. For example, all or any subset of the following may be identified: slope of the surface on which user is walking, whether user is walking or running/dancing/skipping etc., congestion or whether the user is walking straight, the weather, e.g. fair or windy/precipitation.

Operation e. Adjust differences computed in operation c, to compensate computationally for environmental differences between the two walks or compare walks-one without dual-tasking and one with dual-tasking—taken under similar environmental conditions e.g. by filtering out walks that were taken under unusual or different environmental conditions. For example, the system may filter out walks taken on other than a level surface, walks taken in bad weather, walks taken in congested areas or inside, etc.

Operation f. If adjusted differences between gait parameters while dual-tasking and the same gait parameters while walking without dual-tasking, are large relative to end-users' own past norms for such differences, and/or relative to population norms, label the user as suffering from cognitive decline.

When seeking to train a model to estimate Cognitive decline indication all or any subset of the following may be used: previously reported A1c results and CASI scores collected by the apparatus and dashboard as labels. The regression model (say) may be trained to output all or any subset of these scores. Deterioration in gait quality and/or a decrease in gait speed and/or increase in variability and/or dual task cost measures may be used to indicate instability and risk of falls.

According to certain embodiments, operation e is omitted and operation f uses the differences computed in operation c rather than using adjusted differences. Typically, the system may then use, for dual-tasking evaluation purposes, only passive walks which conform to a uniform profile, hence are comparable, for gait parameter comparison purposes, thus may be meaningfully compared in terms of gait parameters, e.g. in both walks (user is dual-tasking and user is not dual-tasking):
i. user is walking not running/dancing/skipping, ascending stairs etc.; and/or
ii. no congestion, thus the user is walking straight rather than weaving between obstacles/other people and/or
iii. weather fair, not windy/precipitation and/or
iv. Similar slope/inclination, e.g. roughly level, both when the user is dual-tasking and when s/he is not.

Thus in the method of FIG. 1b for example, operation b may be followed by all or any subset of the following, in any suitable order e.g. as follows:

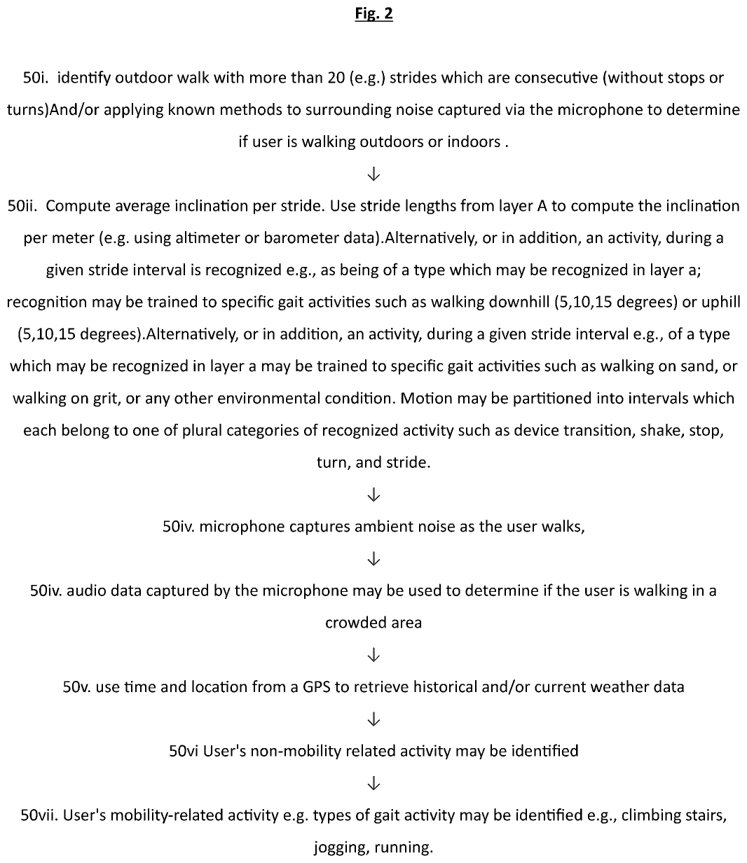

Operation c1. Use IMU data to identify environmental characteristics of the two walks, e.g. slope, whether user is walking or running/dancing/skipping/climbing stairs etc., congestion or is user walking straight, weather (e.g. use all or any subset of operations included in method of FIG. 2)

Operation d1. compare gait parameters characterizing dual-tasking and non-dual-tasking (aka single tasking) walks to one another, after filtering out certain walks with certain environmental characteristics identified in operation c1, as per system criteria determining which walks are to be compared and which are not

Operation f. if differences aka cognitive load, found in operation d1, are large relative to cognitive load norm/s for dual-task vs single-task, label user as suffering from cognitive decline

Any suitable selection scheme may be employed to ensure comparability e.g. by selecting only walks which conform to the above example profile. For example, each time the system is tasked with testing dual-tasking, the system may check a current time interval to determine whether the above criterion i holds; if so the system checks whether criterion ii also holds, and so on for criteria iii, iv. Once an interval has passed all criteria included in the uniform profile (e.g. criteria i, ii, iii, iv above), the system may then determine whether the time interval is or is not a dual-tasking interval. If so, the system may search for an additional time interval which also passes all criteria, and is not a dual-tasking interval. If not, the system may search for an additional time interval which also passes all criteria, and is a dual-tasking interval.

To ascertain whether (i) holds, methods shown and described in co-pending patent document US Publication number: 20200289027 (“System, Method and Computer Program Product For Assessment of a User's Gait”), the entire disclosure of which is hereby incorporated herein by reference, may, for example, be employed to determine which physical activity a user is engaged in during a given passive walk, or at a given time e.g. is the user walking or climbing stairs.

To ascertain whether (ii) holds, the method may use accelerometer/IMU data to identify the user's direction (and then, if the direction aka heading holds steady, but not otherwise, (ii) holds). And/or, the accelerometer data may be used to directly identify changes in heading/direction, and if none of these occur during a given time-interval or during a given passive walk, (ii) holds.

It is appreciated that when an accelerometer in a user's phone measures acceleration, the direction of the force gives insight into which way the phone (hence end-user bearing the phone) is moving. For example, if the user is accelerating forward along the X-axis, the accelerometer may show positive values on the X-axis, whereas if the user changes direction and begins to accelerate along the Y-axis instead (e.g., due to the user having turned left), the accelerometer may record acceleration on the Y-axis.

An accelerometer may directly detect when the phone, hence user, changes direction, because once a user is no longer moving in a straight line and begins instead to change direction, the accelerometer s/he bears may measure forces that are perpendicular to the original direction of motion. For example, given a user who was moving forward, and then turned left: before the turn, the accelerometer registers acceleration along the X-axis (moving forward). During the turn, the accelerometer's readings shift, then, after the turn, the readings re-stabilize.

To ascertain whether (iii) holds, weather reports characterizing a user's known real-world location during a given passive walk, or at a given time, may be employed.

To ascertain whether (iv) holds, barometer data supplied by conventional cellphones may be employed.

Any suitable technology may be used to identify one passive walk interval in which the human is known to be dual-tasking, and to identify a second passive walk interval in which the human is known not to be dual-tasking, e.g. as described elsewhere herein.

Any suitable operationalization may be employed to compute differences between gait during interval1 aka dual-tasking, and gait during some other interval of standard walking with no dual-tasking, such as but not limited to computing a difference e.g. by subtraction, between the values of those gait measures, for example the difference between gait speed in a regular walk and in dual-task.

The output indication may be fed to an external computerized system (including, say, another cellphone) using any suitable technology for sending digital content wirelessly to a remote location, including but not limited to: Wi-Fi, e.g. if both phones have Wi-Fi connectivity; uploading the digital content to a cloud storage service (e.g. Google Drive, Dropbox, or iCloud), and then the external system may access the content via an Internet connection; File Transfer Apps (e.g. as in Shareit, Xender, and AirDroid) for file sharing over Wi-Fi or mobile data for direct transfer (e.g. peer-to-peer) between devices or systems without using Internet data; use of FTP (File Transfer Protocol) e.g. if an external system/device has access to an FTP server at a location which is remote relative to the monitored individual, and has setup and access credentials for the FTP server, the external system/device may upload/download files using FTP clients; use of Bluetooth, for file-sizes and devices that support Bluetooth, to transfer files wirelessly, e.g. between smartphones and tablets within Bluetooth range, e.g. within a single building or campus, Mobile Hotspot technology, e.g. if an external system/device at a location which is remote relative to the monitored individual has no Internet access, but has cellular coverage, a mobile hotspot feature on the device or on a dedicated mobile hotspot device may be used to enable the external system/device to connect to the Internet and access digital content.

It is appreciated that dual-tasking may include any engagement of a person in cognitive and physical activities, simultaneously.

The output indication may include all or any subset of:
a. an audio signal audible to the human bearing the cellphone
b. an automatic telephone call to a subscriber-specific destination e.g. family members of the subscriber whose contact particulars are pre-registered in a system database and/or to a destination common to all/many subscribers, e.g. emergency services
c. the output indication may be integrated automatically into the patient's medical records, e.g. in an EMR (Electronic Medical Records) system run by the patient's registered health provider organization e.g. HMO.
d. an electronic message which is sent to an entity external to the system, or to a human user. For example, a caregiver may receive the output indication on a mobile app such as WhatsApp, or any other messaging app, e.g. as an SMS setup, and access credentials may be used; the system may have a data repository of all caregivers registered in the system including their contact particulars, and an identifier of the user/s for whom they are caring.

Additionally, a caregiver may receive the output indication on a web app designed to monitor plural patients. Any suitable technology may be used to enable system users to connect to the web app, depending on the web app's design and/or the system's architecture. For example, systems provide their users with the web app's URL (web address) e.g. via email, documentation, or a direct link within another system. Users authenticate themselves e.g. by entering a username and password or by using single sign-on (SSO) assuming system integration with services like Google or Microsoft, which provide single sign-on (SSO) functionality. There may be a web app account setup process, e.g. if the web app requires users to create and set up an account, e.g. filling out personal information and setting security preferences. The system may assign permissions and/or roles to users that determine which portions or features or functionalities of the web app each user can access. For example, roles may include all or any subset of: senior care facilities, app stores, health stores, research facilities, mobile phone distributors seeking to harness their technology to improve healthcare, law enforcement authorities, home-care staff, physiotherapists, doctors, family members, or neighbors.

Optionally, certain users may assign, to other users, permissions to access certain features or data within the app, e.g. if users A and B are defined to have a doctor-patient relationship, then Dr. A may be entitled to assign to B or to other patients, permissions to access certain features or data within the app such as Video chat, home exercise program, and specific protocols which may include different measurements and questionnaires.

Users may be prompted to configure settings or preferences within the web app to integrate the app with other tools they use, e.g. their registered health provider organization's EMR, or other wearables, such as smartwatches.

The web app may be part of a larger system, in which case the web app may require integration or connection setup, e.g. linking accounts and/or importing data and/or configuring APIs (Application Programming Interfaces). According to certain embodiments, the system enables each user to sign in with her or his user's account.

Using the Web App then becomes possible; once connected, users may interface with the web app.

Additionally, a caregiver may receive the output indication on a desktop app designed to monitor plural patients. Any suitable technology may be provided for download and installation by system users. For example, users may obtain the app by downloading the desktop app from, say, a distributor or recruiter's website, or an app store. Once downloaded, users may run the installer and follow prompts to install the app on their desktop computer. Upon first launch, users may be guided through an initial setup process typically including entering configuration details, e.g. which care program the user belongs to (remote care/skilled nursing facility), selecting preferences, e.g. how often the user likes to get notifications, or connecting to a server. Any suitable authentication process may be employed e.g. users may not need to log in using credentials such as username and password e.g. if the desktop app supports single sign-on (SSO). The app may or may not require users who do not have accounts to create one, e.g. during installation or the first time they open the app. The desktop app may integrate with other system/s e.g. EMR/EHR in which case users may be prompted to configure settings to connect the app with these other components of the system e.g. by entering API keys, server addresses, or other connection details. The desktop app may help users ensure they have the latest features and security patches, e.g. by providing automatic updates and/or by prompting users to avail themselves of updates each time these become available.

It is appreciated that the system herein may be integrated with external organizations' web-services which are tasked with issuance of licenses to end-users who are qualified to, say, own firearms or drive a car. Such web-services may require their end-users (or perhaps only high-risk end-users such as the elderly) to provide confirmation that they do not suffer from cognitive deterioration; such end-users may be prompted by these organizations to interact with the system herein in order to provide such confirmation e.g. by downloading an app as described above. It is appreciated that once an end-user has onboarded to the system herein, e.g. via a web app or desktop app, the end-user may then opt into other functionalities of the system, such as providing alerts to family members and/or medical service providers, in the event of deterioration of the user's cognitive status, e.g. below a given threshold. This threshold may be fixed by the end user manually, according to certain embodiments, or may be system-determined, e.g. the system may deem a 10% dual task cost to be significant. The system may also count the number of measures with significant deterioration, e.g. each time the system encounters an over-threshold (e.g. over 10%) deterioration in four gait measures such as stride length, cadence, variability, double support, then the system may consider this as a cognitive impairment indication. For use cases such as prompting the clinician to administer further assessment, a suitable protocol and thresholds are described elsewhere herein.

It is appreciated that obtaining consent may be required, e.g. if due to an output indication having been sent to a cooperating licensing organization regarding a certain gun-owner, the gun-owner's permit is rescinded. It is appreciated that external overrides may be provided e.g. the system may be configured to over-ride rescinding of a gun-owner's permit upon medical confirmation from his health provider that the output indication was a false alarm and the gun-owner is still capable of operating fire-arms.

The phone accelerometer may use any known technology to measure gait parameters, such as any technology shown and described in “System, Method And Computer Program Product For Assessment Of A User's Gait”—patents.justia.com/patent/20200289027—Publication number: 20200289027, Publication Date: Sep. 17, 2020 to Naveh et al. which is hereby incorporated herein by reference in its entirety.

Any suitable technology may be used to automatically identify, and/or differentiate between, periods or windows of time in which the human is known to be dual-tasking, as opposed to periods or windows of time in which the human is known not to be dual-tasking. For example, if a user is known to be on a call during time period T then the assumption may be that this user is dual-tasking during this time period. According to certain embodiments the operating system (e.g. Android) may indicate that a user is on a call during a certain time period T. For example, TelephonyCallback is an Android callback class for monitoring changes in specific telephony states on the device, including service state, signal strength, message waiting indicator (voicemail), and others. It is appreciated that “on call” is one of the Android “telephony states” provided via the Android power manager API to developers.

Typically, a user who is on a call is considered to be dual-tasking, whether or not the user is actually talking on the phone e.g. even if the user is merely listening to the call, s/he is considered to be dual-tasking. Typically, if a user is not on a call, it is inconclusive whether the phone-bearer is or is not dual-tasking. The system may provide several criteria for dual-tasking (e.g. on a call, texting etc.), and if a user does not meet any of these criteria, it is assumed that the user is not dual-tasking.

Another possible criterion for dual tasking is that a user's device is in an interactive mode (e.g. screen is on and/or is being used) where “being used” may be defined as a situation where a user is interacting with touch screen e.g. touch screen senses a swipe or press or long-press. It is appreciated that the Android power manager API makes this information available to developers. The Android Power manager API has an indicator of whether a device is or is not in an interactive mode (screen is on and being used), e.g. as described online here: developer.android.com/reference/android/os/powermanager#isinteractive( ). Typically, if the screen is on, but the user is not gesturing, this indicator is on and not off.

Another possible criterion for dual-tasking is that an end user is interacting with her or his smartphone e.g. while walking, including activation of input options, e.g. pressing physical buttons on the phone; this indication is available to Android developers, given that every button has an event when the button is being pressed (and when the button is released).

Another possible criterion for dual-tasking is that voice activity detection indicates that the end user is talking. Libraries-of-the-shelf may be used to implement this criterion e.g. the following online library: pypi.org/project/SpeechRecognition/

Any suitable voice activity detection or speech detection or speaker recognition-process may be employed and may include noise reduction stage, e.g. by spectral subtraction and/or extraction of features or quantities from a section e.g. time-period of an input signal. It is appreciated that access to this input signal is available to developers, given this is a standard audio stream or a file (mp3, wav) and/or classification to label the time-period as including or not including speech (speech or non-speech), e.g. because certain features exceed certain thresholds, or because a logical combination of conditions (each of which may be true when a certain feature exceeds a certain threshold) is true vs false.

If speaker recognition is used, the system typically pre-stores each end-user's voice signature. For example, when logging into the system, or as part of the onboarding process, end-users may be prompted to record their voice signature as a reference. More generally, onboarding of each end-user may include all or any subset of: obtaining consent to record their audio/speech, recording a voice signature, and/or providing contact particulars of family members and/or health service providers.

Many variations are possible.

For example, any operation described herein as being “continuous” may alternatively simply be automatically performed repeatedly, e.g. according to a programmed schedule, or daily, or periodically.

Also, any embodiment herein may be implemented using typically continuous measurement of passive gait aka background walks, where “passive” refers to “measurement of gait which does not rely on user cooperation” e.g. walk (or some other physical activity) which is being measured, and the measurement was not initiated by the patient, but is instead recorded in the background.

It is appreciated that any suitable process may be used to determine which environmental conditions exist during a given passive walk measurement, and how these environmental conditions are to be taken into consideration when comparing walks taken during differing environmental conditions, e.g. as described herein. It is appreciated that any suitable processor (on-device or remote through an Internet connection) may be used to extract suitable gait analysis from the inertial data, aka IMU data, generated by an end-user whose gait is being recorded by the IMU of the phone or other device sh/e bears as s/he walks e.g. as described in co-owned US patent documents U.S. Ser. No. 17/666,180 published as US 2023/0137198 on 4 May 2023, U.S. Ser. No. 16/659,832 published as US 2020/0289027 on 17 Sep. 2020 and U.S. Ser. No. 17/500,744 published as US 2022/0111257 on 14 Apr. 2022, the disclosures of all of which are hereby incorporated herein in full by reference.

This motion analysis, aka gait analysis, may, for example, yield all or any subset of the following:
a. “motion semantic segmentation” over time including partitioning motion into intervals which each belong to one of plural categories of recognized activity e.g. all or any subset of the following five categories:
   Device Transition—change of the position of the measurement device
   Shake—some unrecognized significant movement
   Stops—the measurement device is stationary; it may be that the subject is standing in place, or that the measurement device is placed aside.
   Turns—the subject is turning or changing course (direction of movement) between adjacent strides significantly (e.g. more than 30 degrees).
   Strides—detection of linear gait activity. Any repetitive mobility activity is considered as gait, such as walking, running, stair negotiation, going up/downhill, cycling (as described in Ser. No. 16/659,832 inter alia). Each stride (or cycle) is attached with its estimated traveled distance (or stride length);
b. the subject's heading over time, relative to the north and/or relative to the altitude above the sea level; and
c. the measurement device's bodily position or placement over time; the system may assume the device does not change its position if no “device transition” interval is detected.

The system may then fuse all or any subset of the above layers a, b, and c (all described in U.S. patent application Ser. Nos. 16/659,832, 17/500,744, 63/272,839 published as US20230137198, the entire disclosures of which are hereby incorporated herein by reference) and the other sensors' data over the course of time. From this fused data, the system may then extract environmental conditions from background walks or passive walks, such as all or any subset of the following environmental conditions: whether walk is taking place outdoors or indoors, incline/surface level, type of surface user is walking on, whether the area is congested, and/or polluted and/or poorly maintained, in what weather the walk is taking place (typically for at least outdoors walks), whether user is engaged (also) in non-mobility activity such as communicating via the phone, and whether the mobility activity characterizing the walk is in fact walking, or whether perhaps the user may be, say, skipping or running, or climbing stairs, or dancing.

Any suitable method or approach may be used to extract all or any subset of the above environmental conditions or aspects (e.g. in the context of operation d in FIG. 1a or operation c1 in FIG. 1b), such as all or any subset of the operations of FIG. 2 described below.

It is appreciated that any suitable semantic segmentation method may be used herein for identifying intervals characterized by, say, device transition vs. shake vs. stop vs. turn, vs. stride/gait e.g. as described in co-owned U.S. patent application Ser. No. 18/975,890 “Advanced Pedestrian Navigation Based on Inertial Gait Analysis and GPS Data”, incorporated by reference herein in its entirety, of which this application is a continuation-in-part and/or in co-owned application US 2022/0111257 entitled “Efficient System Configured To Facilitate Physical Rehabilitation”, incorporated by reference herein in its entirety.

Mobility measures may be specific to certain contextual conditions. For example, passive walk gait speed may be considered a different measure than active walk gait speed because awareness may affect gait and may be viewed as cognitive load and to accurately estimate how well a user copes with dual-tasking, it is desirable to compare dual-task with non-dual-task (aka single task) measurements under contextual/environmental conditions which are as similar as possible. It is appreciated that the terms “contextual” and “environmental” may be interchanged herein.

Alternatively, or in addition, environmental conditions may be derived from user input to ensure that walks with or without dual-tasking which are compared with one another have contextual/environmental conditions which are as similar as possible e.g. by measuring an intentional dual-task walk and/or derivation from motion data. Resulting individual mobility measures (active vs. passive and/or different environmental conditions) may be filtered individually for outliers and/or disrupted data and interpolated individually, rather than being filtered/interpolated together. e.g. by performing fusion (any suitable combination) of Gait data layers a and/or b, and/or c) and/or data from other sensors (such as altimeter, microphone/speaker, weather reports) over time. and then using the fused data layers to extract environmental conditions from background walks e.g. as per operations 50i-50vii herein. For example, audio data received from a user's phone's microphone may be used to understand whether given walks taken by the user occurred outdoors/indoors, and/or whether in a crowded area or not and/or altimeter data may be used to understand an inclination Or slope of a surface on which the user was walking, etc.

For example, walks may be divided into dual-tasking walks, when performing non-mobility activity simultaneously and paying cognitive load while walking, and non dual (aka single)-tasking walks. The same mobility measures collected under different contextual conditions may be considered as different measures. To filter outliers and disrupted data given there may be some discrepancies in the estimated mobility measures, especially in passive gait measurements, since environmental effects may be unknown thus reducing noise, outlying data points may be filtered out (or at least indicated to have a lower certainty or lower degree of confidence). For example, if a patient's timeline is available, comparison of data points and the patient's reference is possible. Thus, for example, a gait measurement with a gait speed higher or lower by three (say) standard deviations from the patient's weekly (say) average gait speed (say) may be considered an outlier. When many data points are available, as in passive gait monitoring, a daily or weekly (or any other timespan) aggregation, taking the median or the average, may also reduce outliers' noise.

According to certain embodiments, the system addresses all environmental conditions, including dual-tasking vs non-dual tasking (aka single tasking), and considers every contextual condition individually. It is appreciated that in practice, “same” mobility measures may describe slightly different versions, and hence different mobility measures.

It is appreciated that all of data layers a, b, and c (aka layerA, layerB, layerC respectively) e.g. as described in co-owned U.S. Ser. No. 18/975,890 filed Oct. 12, 2024, the entire disclosure of which is hereby incorporated herein by reference) may be fused e.g. aligned over the course of time, to yield a single timeline with data layers A and/or B and/or C and data from other sensors if available and gait parameters. Operations 50I, 50II, . . . may then be performed. However, alternatively, layer b may be omitted, and layer a only may be employed. Layer c may be employed to implement operations 50vi onward.

Data layer b aka layer 30b typically includes a data layer storing subject's heading over time, in a plane e.g. relative to the north and/or altitude relative, say, to sea level (typically available from the user's devices' magnetometer and altimeter respectively).

Data layer c aka layer 30c typically stores measurement device's bodily position or placement over time. Any suitable method may be employed to identify bodily position (e.g. right hip pocket, left hand) in which the phone is located e.g. using the 2-way activity/bodily position classifier described in co-owned US20200289027A1), the entire disclosure of which is hereby incorporated herein by reference. If the classifier outputs a bodily position, the system may then assume the user's device's position remains unchanged until a “device transition” interval is (next) detected. The classifier may then be reactivated each time a “device transition” interval is detected.

The method typically makes use of a data layer A aka layerA aka “motion semantic segmentation layer” described in co-owned U.S. Ser. No. 18/975,890 filed Oct. 12, 2024, the entire disclosure of which is hereby incorporated herein by reference, which may be generated by gait analysis over time e.g. in accordance with any of the embodiments of co-owned published U.S. patent application Ser. Nos. 16/659,832, 17/500,744, 63/272,839, the disclosures of which are hereby incorporated by reference in their entirety, including partitioning motion into intervals which each belong to one of plural categories of recognized activity, e.g., all or any subset of the following five categories:
a. Device Transition—change of the position of the measurement device e.g. phone with accelerometer
b. Shake—significant movement which is unrecognized (e.g. not falling into any other class); this is characteristic e.g. of a cellphone deployed in a moving vehicle. Typically, if a user moves his phone while he is turning, the class is SHAKE rather than TURN, because the net motion is not that of a turn, and if a user moves her phone while she is standing in place, the class is not STOP but SHAKE, because the net motion is not that of a stop.
c. Stops—the measurement device is stationary, possibly indicating that the subject is standing in place or that the measurement device has been set aside and is not presently on the user's body.

This may be identified as velocity zero, however it may be difficult to estimate velocity accurately, directly from IMUs. Alternatively or in addition, if for a period of time accelerations are static or very low e.g. less than 0.05 m/sec**2, this may be used to indicate that the sensor is static, or does not accelerate.
d. Turns—the subject is turning, or changing course, or changing heading, or changing direction of movement. Typically, gyro readings may be used to yield a relative change in heading from one stride to another; a heading change between adjacent strides may be regarded as significant if it is over a threshold (e.g. more than 30 degrees).
e. Strides/gait—detection of linear gait activity. Any repetitive linear mobility activity may be considered as gait, such as walking, running, climbing stairs, going up/downhill, cycling (as described in co-owned published U.S. Ser. No. 16/659,832 inter alia, the entire disclosure of which is hereby incorporated herein by reference). Each stride (or cycle) is typically attached to or associated in memory with its estimated traveled distance (or stride length) and/or other gait measures corresponding to each stride, such as but not limited to all or any subset of the following: cadence, cadence variability, double support and single support of either leg, stride length asymmetry, and stride width.

The method of FIG. 2 may include all or any subset of the following operations, suitably ordered e.g. as follows:
50i. A segment of walking is considered outdoor walking if it has more than 20 consecutive strides. Any other walk segment containing stops or turns is considered indoor walking. Alternatively, or in addition, another approach is adding/using the surrounding noise captured via the microphone to determine whether the user is walking outdoors. (www.sciencedirect.com/science/article/pii/S2405959515300795)
50ii. Use altimeter data to measure the change in altitude (aka surface level) over time, measure the altitude difference of each stride, and compute the average inclination per stride. Use stride lengths from layer A to compute the inclination per meter.

Alternatively, or in addition, an activity, during a given stride interval e.g., of a type which may be recognized in layer a e.g. as described in U.S. patent application Ser. Nos. 16/659,832, 17/500,744, 63/272,839 published as US20230137198, the entire disclosure of which is hereby incorporated herein by reference; recognition may be trained to specific gait activities such as all or any subset of walking downhill at various declines (e.g. 5, 10, 15 degrees) or uphill at various inclines (e.g. 5, 10, 15 degrees).

Alternatively, or in addition, an activity, during a given stride interval e.g., of a type which may be recognized in layer a e.g. as described in U.S. patent application Ser. Nos. 16/659,832, 17/500,744, 63/272,839 published as US20230137198, the entire disclosures of which are hereby incorporated herein by reference, may be trained to specific gait activities such as walking on sand, or walking on grit, or any other environmental condition. It is appreciated that motion may be partitioned into intervals which each belong to one of plural categories of recognized activity such as device transition, shake, stop, turn, and stride/gait.
50iv. The microphone captures ambient noise as the user walks, and the audio data captured by the microphone may be used to determine if the user is walking in a crowded area, e.g. as described in the following online publication: ris.utwente.nl/ws/portalfiles/portal/220586486/Wang2020sound_based.pdf, the entire disclosure of which is hereby incorporated herein by reference.
50v. The system may use time and location from a GPS to retrieve historical and/or current weather data (openweathermap.org/history)
50vi User's non-mobility related activity (which user may be engaging in while walking, in which case this is evidence of a dual-task walk) may be identified, for example.
50vi-a. User-smartphone activities, such as listening to music, interacting with the phone, and talking over the phone (all may be determined using the smartphone OS standard APIs); these may be regarded as evidence of dual-tasking if the user is also exhibiting mobility-related activity and/or
50vi-b. The user is talking/speaking (not over the phone)—using the sound captured via the microphone to determine if the user is speaking (docs.nvidia.com/nemo-framework/user-guide/latest/nemotoolkit/asr/speaker_recognition/results.html#speaker-verification-inference). Verification requires a baseline speaker recording which takes place at the patient onboarding. The system then takes the baseline and current audio recordings, and may then, as described above, verify that the patient is speaking, which again is evidence of dual-tasking if the user is also exhibiting mobility-related activity.
50vii. User's mobility-related activity—using activity recognition functionality e.g. as described in U.S. patent application Ser. Nos. 16/659,832, 17/500,744, and 63/272,839 published as US20230137198, the entire disclosures of which are hereby incorporated herein by reference, typically trained to identify, per stride interval e.g., specific gait activities being performed such as climbing stairs, jogging, running, and any other form of specific gait activity.

In active cognitive impairment evaluation, the comparison is typically between a regular walk measurement, immediately followed by an dual-task walk measurement. The two walks may be performed immediately, one after the other, to minimize any other influence on the patient's walk other than the dual task instructed. The difference in gait parameter values (in the less-favorable direction) is considered the dual-task (or cognitive) cost.

It is appreciated that the dual-task paradigm may include the following:
1. Pre walk (single task walk)—The patient walks for 45 secs (say) at a comfortable pace.
2. Dual task walk—The patient walks for 45 secs at a comfortable pace performing a cognitive task e.g. counting backward or verbal fluency, while walking.
   Once both walks are completed, a score may be presented, and next steps e.g. recommended call/s to actions where score computation may, for example, use formulae described elsewhere herein.

If the test results are positive, a recommendation to proceed with further assessment may be displayed, suggesting to choose between cognitive screening tools e.g. SLUMS or MMSE tests.

If the test results are negative and there is no indication of cognitive decline, a recommendation may be provided, e.g. to re-evaluate in one month.

However, for patients using personal phones equipped with an app providing any functionality shown and described herein, gait may be measured, typically passively, both within the app and in the background, continuously tracking for changes and potential decline. If a decline in gait parameters is detected, the patient may be advised to undergo the dual task cognitive assessment protocol with their clinician to evaluate the extent of cognitive decline. Comparison may be between the patient's baseline gait assessment vs. the last aggregated assessment in the listed gait parameters. The baseline is computed by averaging the first screening and passive measurements of the first week. The last aggregated assessment is composed of the average of the last week's measurements.

Any suitable method may be employed for monitoring cognitive impairment and generating cognitive decline indications on the dashboard. Typically, all or any of the following apply:
a. The dashboard shows the clinician's patients
b. Each patient is presented with the measurement status, which is required by the organization's protocol
c. In addition, the two types of cognitive impairment and cognitive decline indications are shown for any patient who has been diagnosed with any of them
d. The relevant call to action appears in the rightmost column
e. This screen presents the status of patients who either performed a cognitive screening in the supervised setting or were monitored remotely.

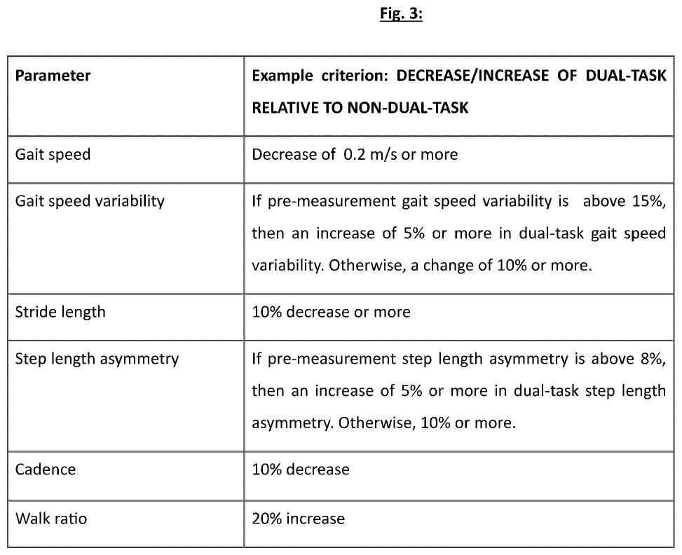

Also, any suitable thresholding may be employed to determine when to alarm. For example, any of the criteria shown in the table of FIG. 3, even standalone, or any logical combination of the following, may be used as criteria or formula determining whether or not to alarm:

And/or, according to some embodiments, a phone-bearer may be prompted at least once to electronically provide consent, via his phone, and/or cooperation to conduct a dual-tasking test which may involve user cooperation (as opposed to unobtrusive “background” or “passive” testing). And/or, according to some embodiments, patients visit their health-provider's clinic, and para-medical personnel (e.g. nurse, assistant, medic) may use a health monitoring app on a clinic's cellular phone, borne by the patient undergoing the test, thereby to evaluate plural patients using a single cellular phone, without downloading any apps onto on the phones of, say, elderly persons who may not have their own phones. And/or, according to some embodiments, an automatic call is made to a phone-bearer at home, to obtain consent for either conducting a dual-tasking test which may involve user cooperation (as opposed to unobtrusive “background” testing), or for re-scheduling the test for a later time.

Also, any suitable process may be used to test cognitive decline using a mobile phone's sensors. For example, for “active” testing, the process may include all or any subset of the following operations, suitably ordered e.g. as shown:
a. prompt end-user to grant access to phone's microphone, to the functionality e.g. app of the present invention. This enables the app to record the end user's speech and/or analyze the performance level of the dual task.
b. select current phone location on the body e.g. in pants pocket, on strap affixed to body, in skirt pocket, in shirt pocket, etc. where the phone may remain during the dual-task period and no-dual task period. This may be done by displaying illustrations of these locations and asking the user to select the appropriate one.
c. prompt the user to perform a (typically user-selected) cognitive task when a countdown finishes e.g. “count out loud from 100 downward, subtracting 3 each time” or verbal fluency e.g. “recall animal names that start with the letter ______”
d. prompt user to start the app's countdown when s/he is ready
e. sensors measure gait parameters directly or indirectly
f. system determines “cognitive cost” of dual task by determining differences between gait parameters in first period (no dual task) and second period (gait while doing dual task) e.g. differences (in m/s and/or %) in gait speed; different in stride length (in m/s or %), difference in step length asymmetry (e.g. in %), cadence (e.g. in rpm or steps per minute), walk ratio (relationship between the amplitude and the frequency of the movements of the legs—e.g. in %), distance walked consistency of gait, etc.
g. system combines cognitive cost data, e.g. all or any subset of the above differences using any suitable logical and/or computational combination formula, and if the combined value exceeds a threshold, it generates an alarm.

Also, any suitable process may be used to passively test cognitive decline using a mobile phone's sensors e.g. all or any subset of the above operations, suitably ordered e.g. as above.

It is appreciated that operations a-g above may somewhat differ, depending on whether detection of cognitive status is active or passive. For example, there may or may not be a final, return-to-start operation, namely continuing measurement continuously, and/or, in passive measurement, the system typically cannot elicit the end-user's input such as phone's position, and cannot instruct the end-user to perform or not perform a dual task. Instead, automatic inference may be used, e.g. as described elsewhere herein in the context of extraction of environmental conditions.

Also, the output signal may be fed to any suitable external computer system e.g. all or any subset of the external systems listed below. For example, some computer systems help general practitioners (GPs) manage patients and generate alerts for upcoming appointments, follow-ups, or patient care reminders. Such systems include but are not limited to Electronic Health Record (EHR) Systems, Practice Management Software, which may themselves integrate with EHR systems, Clinical Decision Support Systems (CDSS), and Patient Relationship Management (PRM) systems. It is appreciated that some systems, e.g. EHR and practice management software, may have built-in alerting and notification features; according to certain embodiments these are customized to send alerts to GPs and/or staff responsive to output indications generated by the system shown and described herein. Logical conditions for sending alerts (e.g. for follow-ups or preventive care) may combine output signals generated by the system shown and described herein with other criteria supplied by the external system, e.g. patient data such as abnormal test results, clinical protocols, and patient demographics. Alerts may even be integrated directly into a health provider's workflow dashboard.

Thus, the external computer systems to which output signals may be fed may include all or any subset of the following state-of-the-art systems serving GPs: Epic, Cerner, Allscripts, Athenahealth, Meditech, NextGen Healthcare. Such systems may themselves integrate with other healthcare provider systems.

Any structured and secure method for communication may be employed e.g. establishing connections through a Health Information Exchange (HIE) or any other secure electronic system that allows data, e.g. alerts, to be shared electronically. Standardized Messaging Protocols e.g. HL7 (Health Level Seven) may be used to ensure interoperability of systems, or any other standard configured to define structure and format of messages provided to healthcare information systems, including alerts. Communication may be compliant with healthcare data privacy regulations, such as but not limited to HIPAA. Data, e.g. alerts, may undergo encryption during transmission and may be subject to suitable authentication and access control schemes. Alert Delivery Confirmation schemes may be employed to ensure alerts have arrived safely.

Alert notifications generated by the system herein may be integrated into workflows of healthcare providers receiving the alerts, e.g. by displaying alerts within providers' EHR system interface or integrating alerts into clinical decision support tools. Alternatively, notifications may be sent to providers via secure messaging platforms.

It is appreciated that responsive to (inter alia) output indications provided by the system shown and described herein, any or all of the following may be scheduled for a cellphone bearer who is found to comply with criteria indicative of low cognitive status and/or cognitive status decline, aka cognitive decline: Consult with Healthcare Professional for cognitive assessment, testing (e.g., blood tests, brain imaging) to augment diagnosis, automatic generation of a care plan including recommendations for treatments and/or medications and/or lifestyle modifications and/or support services and/or diet and/or exercise and/or activities that promote cognitive health; assessment of a person's safety at home and in daily activities to determine whether hazard removal or other modifications are to be made to the person's home environment to reduce risks of accidents, falls or wandering; installment and/or deployment of safety device/s and/or monitoring device/s; increased supervision; cognitive-status appropriate dental care, given that conventional dental care requires and assumes patient cooperation, which becomes less and less available as cognitive status declines.

It is appreciated that certain types of malfunctioning correlate with cognitive decline, such as incontinence or difficulty in swallowing; thus improved, cost-effective, continuous detection of cognitive decline may also improve the healthcare system's handling of these malfunctions, as close as possible to when they develop; this may, for example, prevent adverse effects of choking due to difficulty in swallowing, which may even include death.

The terms mobility measure, motion parameter, and gait parameter may be interchanged herewithin. For example, gait parameters are intended to include standard measures describing an individual's walk (such as but not limited to speed, cadence of steps per minute, stride length, width, stance time, right & left). Mobility measures may (also) include parameters which are not gait parameters, e.g. parameter/s characterizing turning, standard test performance (such as but not limited to TUG time, standing/sitting durations), balance measured by the magnitude of movement or duration of stability during static standing test (Romberg).

The terms caregiver and clinician may be interchanged herewithin.

The terms “accelerometer” and “IMU” may be interchanged herewithin.

### Example Implementation

The system may include all or any subset of a caregiver-facing dashboard, a patient-facing mobile app aka Patient app, and a digital care web service that typically communicates with the patient app and the caregiver dashboard. Typically, the patient-facing mobile app enables the system to collect data from the patient, as well as communicate back to the patient, to provide clinical value and encourage the patient to be engaged and adhere to their care program. The digital care web service is typically configured for communication with the two other components, which enables their functionalities and/or is tasked with analysis of data collected from the end-user, e.g. patient.

Also, administrative tools may be provided e.g.:
1. Registering a patient to the system
2. Moving a patient from one clinic to another or from one clinician to another
3. Adding information to a registered patient

The patient app typically serves as an endpoint of the system on the patient's side which enables the system to collect data from the patient and/or enables the patient to understand their status over the treatment and/or communicate with their caregiver and/or consume their caregiver's treatment program at home. Example embodiments for the collection of patient data are now described.

### Collection of Patient Data

The patient app may receive all or any subset of the following:
1. Objective gait analysis which may be performed on raw data collected by the end user's phone's accelerometer and may include all or any subset of the following measures:
a. Spatiotemporal analysis: evaluation of gait properties such as gait cycle time, cadence, gait speed, stride length, right and left step length, base width, right and left single support and stance time and percentage, double support time and percentage.
b. Kinematics: including any of the lower body joint angles over the gait cycle
c. Kinetic: evaluation of gait properties related to torques and forces such as ground forces and center of feet pressure over the gait cycle, and the analysis of its sway over the gait cycle
d. Variability: the deviation of any of the gait properties above between different strides over the same walk. May be processed in any suitable form, e.g. aggregated as the standard deviation of each value and/or as a sequence of the differences in values over time.
2. Mobility tests which may include an objective assessment of standard functional tests, for example all or any subset of the following:
   a) TUG—www.physio-pedia.com/Timed_Up_and_Go_Test_(TUG)
   b) Sit to stand—www.physio-pedia.com/30_Seconds_Sit_To_Stand_Test
   c) Rhomberg balance test—www.physio-pedia.com/Romberg_Test
   d) Four square step test—www.physio-pedia.com/Four_Square_Step_Test
   e) The 4-Stage Balance Test—www.physio-pedia.com/The_4-Stage_Balance_Test
   f) Reaction time in walking—the user is asked to walk back and forth and make U-turns (right or left) according to cues signaled by the device. The time between the cues' timestamps and the corresponding turns' timestamps is then averaged and evaluated as the user's reaction time.
3. Subjective reporting of the patient which may include questionnaires and patient-reported outcome measures, such as all or any subset of the following:
a. The Falls Efficacy Scale—www.physio-pedia.com/Falls_Efficacy_Scale_-_International_(FES-1)
a. The Lower Extremity Functional Scale www.physio-pedia.com/Lower_Extremity_Functional_Scale_(LEFS)
a. The WOMAC Osteoarthritis Index—www.physio-pedia.com/WOMAC_Osteoarthritis_Index
a. The Oswestry questionnaire—www.physio-pedia.com/Oswestry_Disability_Index
a. www.physio-pedia.com/36-Item_Short_Form_Survey_(SF-36)

Possible implementations of all these three above functionalities are described in co-owned U.S. Pat. No. 11,751,813) Entitled “System, Method And Computer Program Product For Detecting A Mobile Phone User's Risky Medical Condition”, and in Processing a Mobile Phone User's Condition) to Davidson et al, published on Nov. 1, 2024 under publication no. US 2024/0008766, the entire disclosures of which are hereby incorporated herein by reference.

### The Caregiver Dashboard

Typically, the caregiver dashboard allows the clinician to take different actions of care based on the patient's data for the individual patient and/or enables management of clinical resources such as clinician attention and triage of patients in the scope of a clinic having a group of patients. And/or a clinic manager or an organizational manager may compare different clinics in terms of mobility assessment of their patients in order to make organizational decisions. These three (say) levels of data-driven decisions are elaborated on in the section on the caregiver's dashboard.

Caregiver dashboard functionalities may include all or any subset of the following:
1. Start assessment
2. Gather all patient information e.g. all or any subset of:
a. Mobility
a. Norms
a. Subjective
a. Clinical events
a. HEP, aka Home exercise plan. A physical therapist or caregiver may prescribe exercises for a patient at home, and may get feedback on completion/execution of that program.
3. Assess patient's current status e.g. cognitive decline, optionally in conjunction with all or any subset of: fall risk status, change in mobility vs. onboarding, surgery, last month . . . , comparison to their reference population, recovery pace, visual deterioration.

Typically, the caregiver dashboard provides the clinician with administrative tools and/or triaging tools on plural patients, and/or recommended care actions for the individual patient.

Care actions may be defined in all or any subset of the following three levels: individual patient level, plural patients level, organizational level.

Care actions for the individual patient, all of which are typically data-driven, e.g. derived at least party from gait/mobility data collected from the patient's cell phone's accelerometer, may include all or any subset of: Alerting on patients at severe risk—e.g. to ensure that a suitable clinician sets up an appointment with the patient in question, or otherwise contacts that patient. Such actions include:
1. Prompt the patient to provide data
2. Justification of a care program e.g. by data-driven selection thereof—make data-driven decisions for efficient care delivery
3. Justification of extension of care (e.g. typically defining a duration, and, accordingly, extending a current plan)
4. Review patient's data—review patient progress, risk profiles, feedback, and mobility scores
5. Communication with the patient—communicate effortlessly or build a remote plan of care, for example:
   1. Get intervention suggestions based on patient data
   2. Home exercise program with a set of templates and auto-assignment
   3. Show patients their progress and perform gait training with ‘live feedback’
6. Documentation—strengthen documentation with objective data, for example:
   1. Generate progress notes and reports in seconds
   2. Justify medical necessity and secure reimbursement
   3. Seamlessly integrate results into existing EMRs
7. Assign and adjust personalized services e.g. video exercise programs, for example:
   1. Pre-built and customizable protocol templates
   2. Smart suggestions based on patient behavior and best practice
   3. Option to upload unique exercise and education videos
8. Distribution of any type of equipment; typically distributing a first hardware item such as a wheelchair, and a second hardware item such as an oxygen tank, are defined in the system as separate care actions.
9. Performance of any type of service or treatment/s; typically administering first or second treatments are defined in the system as separate care actions.

It is appreciated that dual-task is not the only cognitive status assessment option when using phone accelerometers to monitor gait. For example, cognitive decline may be associated with a decline in gait speed (as established by researchers such as Callisaya et al. 2015; Deshpande et al. 2009; Atkinson et al. 2007). Other gait measures such as pace, rhythm, and variability have also been identified as predictive of future risk of cognitive decline and dementia e.g. by Verghese et al. 2007 and Pieruccini-Faria et al. 2021. Also, spatiotemporal measures of gait and greater variability of gait parameters have been found to be associated with and predictive of both global- and domain-specific cognitive decline such as memory, executive function, visuospatial, and language (as indicated e.g. in a Savica et al. 2017 report of a Mayo Clinic study conducted on over 3,000 participantsl.

Typically, a caregiver's tools, e.g. ranking of patient for triage, alerts, generating list/s for further assessment/examination) depend on the mobility assessment of the individual and the plural patients. According to certain embodiments, motion analysis is provided which includes gait and/or mobility measurement using a mobile app on a single device with IMU; the system may include a patient's app, and/or caregiver workflows which may be presented on a caregiver dashboard and/or monitoring processes which may take place in a digital service component. Any suitable method may be employed to implement these functionalities, including presenting content, e.g. output data of monitoring processes which may take place in a digital service component, to the caregiver and/or patient in the dashboard or app.

It is appreciated that the environmental/contextual conditions described herein are merely by way of example. Any suitable environmental/contextual condition may be included in similarity criteria which determine whether or not a given walk is to be used for comparison purposes. For example, a classifier may be trained to identify whether a person is carrying nothing, or is carrying an object weighing up to 1 kilo, or 1-3 kg, or 3-7 kg, or 7-plus kg. then, only walks by persons falling within, say, the first class are compared with other walks, to ensure similarity. Or, a classifier may be trained to identify whether a person is or is not holding hands.

It is appreciated however that insisting on very close similarity between walks to be compared may result in insufficient availability of data if some or all users only infrequently take walks which answer to system similarity criteria. For example, if the similarity criteria under which walks are compared (vs discarded walks which are deemed insufficiently similar and thus are not compared) includes similarity over a large number of parameters e.g. weather, incline, weight being carried, cane yes/no, holding hands yes/no, indoors/outdoors, dark/light and more, and/or, say, that walks to be compared must be along surfaces whose inclines fall within only 2 degrees of one another as opposed to 15 degrees of one another, this insistence on very close similarity may result in insufficient availability of data if, say, many users only infrequently take walks along precisely such inclines. As the system matures, the system may relax or tighten similarity conditions to ensure that environmental/contextual conditions are as uniform as possible without, however, resulting in insufficient availability of data. When relaxing a certain similarity criterion, the system may check population norms or sub-population norms to confirm that relaxing the criterion does not result in any artifactual differences in gait parameters which are large in comparison to the effect of cognitive load on gait parameters (which may for example be pre-tested for a sample of, say, 60 persons, by comparing dual-tasking to single-tasking under identical conditions). For example, the system may compute various gait parameter averages for walks taken by the population up a 15 degree incline vs a zero degree incline, to determine whether a similarity criterion demanding that walks to be compared be along an incline whose slope is from zero to 10 degrees, may be relaxed to demand only that the walks to be compared be along slopes of less than 15 (rather than 10) degrees.

Also, as the system matures, the system may define criteria for walks to be compared that differ from person to person. For example, if for some users, the weather is almost always rainy, the weather criterion enabling walks to be compared may, for that person, be defined by the system to be rainy weather. Or, if for some users with limited mobility who live on a 16 degree hill, walks are almost always either uphill walks up a 16 degree incline or downhill walks down a −16 degree incline, the slope criterion enabling walks to be compared may, for that person, be defined by the system to be the union of those 2 values (plus or minus 15 to 17 degrees e.g.).

It is appreciated that the system typically sets out to measure cognitive load frequently e.g. daily; on some days, no measurements may be available e.g. because the user did not walk on that day, or because the user's walks did not conform with the system's criteria qualifying walks to be compared e.g. because the user walked only in the rain whereas the system's criteria qualifying walks to be compared demand fair weather or because the user walked only in a congested areas whereas the system's criteria qualifying walks to be compared demand a non-congested area.

Each day, the system may for example perform gait analysis according to any embodiment herein once per minute or once per 5 minutes, say, typically passively or in background or without alerting the user or without enlisting user cooperation, on an imu data sample which is, say, one minute long. If the sample is not “gait” at all, or if the gait does not answer to a system criterion e.g. is along a steep incline or is in a congested area, the sample is discarded. As soon as the system has identified a sample which is gait e.g. is a passive walk sample, which additionally answers to system criteria, the system may determine whether the sample is dual-tasking or single-tasking. Once a dual-tasking sample (say) has been identified), the system may then analyze only single-tasking samples or vice versa, once a single-tasking sample has been identified), the system may then analyze only dual-tasking samples. Once a pair of samples has been found, which are passive walk samples, one dual-tasking which occurred say at 947 am, and the other single-tasking which occurred say at 1513 pm, both of which answer system criteria for walks to be compared, the cognitive load may be computed by comparing gait analysis outputs from the two samples and may be stored, time-stamped for that day.

According to certain embodiments, pretesting is performed to determine what size of effect is caused by each of various components of the similarity criterion. For example, pretesting in laboratory conditions may determine that, for certain population subsets, even small differences in, say, wind conditions significantly affect gait parameters such as speed or cadence, whereas, say, slopes within the range of zero to 5 degrees hardly affect these gait parameters at all. these effects may, during pretesting, be compared to the size of the effect of dual-tasking itself e.g. be compared to the differences in the same gait parameters which occur in single vs. dual-tasking, e.g. to ensure that differences between those walks that fall within the similarity criterion generate effects on gait parameters which are small relative to the effect of single vs. dual-tasking.

It is appreciated that cognitive evaluation functionality herein is useful for a very wide variety of tasks which today are performed less cost-effectively, less accurately at least because of lesser frequency, and inefficiently because the unifying component, namely the need to factor in a well-estimated and current estimate of cognitive functioning, which may be based on end-user data collected once for several or many different purposes, e.g. as described herein, is not recognized.

For example, in Israel, general practitioners require cognitive evaluations of their patients in order to provide proper care; in parallel, government licensing agencies provide (or do not provide) licenses for hiring foreign workers for eldercare, based in part on cognitive status which may be evaluated qualitatively rather than objectively, and dental care public policy agencies have little, if any, data, hence solutions, available regarding prevalence of, or trends of, cognitive decline in the population, although such declinate impacts the patient's ability to cooperate which is pre-requisite for accepting dental care.

Another example is that diabetic ketoacidosis (DKA) is frequently associated with altered mental status thus daily (say) cognitive evaluation using the dual task paradigm as described herein, may be useful in early detection of DKA in a general or diabetic population not known to suffer from DKA, e.g. by prioritizing individuals showing reduced mental status for clinician attention and generating automatic reminders for such individuals' clinicians, to prompt clinician to identify or rule out DKA. This embodiment may be employed e.g. in conjunction with any system described in co-owned U.S. application Ser. No. 19/049,400 filed Feb. 10, 2025, the disclosure of which is hereby incorporated herein in its entirety.

On the collective level, embodiments herein provide an improved method for estimating the need for hardware and/or services and on the individual level, and/or an improved method for estimating when individuals are to transit from a need for one type of hardware, to a need for another. It is appreciated that cognition affects even physical needs, e.g. because the demented individual cannot cooperate. For example, an individual may be physically capable of moving from wheelchair to car, however due to his or her cognitive status, the individual may not cooperate with that move, necessitating (for any needed mobility from place to place) a wheelchair-accessible van which may include a ramp and/or or a lift and/or lowered floor, space to accommodate a wheelchair, and tie-downs to secure a wheelchair safely while the vehicle is in motion. Other costly and space-consuming hardware, a need for which may be affected by cognitive status, includes all or any subset of: motorized wheelchairs, shaftless elevators, chairlifts for moving between floors, leg lifters, hoists, pool lifts, hoists such as ceiling hoists, floor hoists, gantry hoists, sit to standing hoists, and patient lifting hoist slings.

It is appreciated that embodiments herein also enrich data-gathering functionality of mobile phone manufacturers and/or distributors and/or data brokers, assuming a suitable scheme for handling privacy concerns is provided, e.g. an automated consent scheme for an optional opt-in in return for reduced fees. Such data is useful for pharma enterprises tasked with the development of new pharmaceuticals, for example.

It is appreciated that embodiments herein provide (full care action selection and/or) decision support for health providers generally, e.g. regarding care action parameters such as but not limited to when to discharge patients from, or extend care duration in, care frameworks such as mental health facilities and/or supervised living and/or out-patient psychiatric or neurological follow-up, and/or transition from care framework a to b or c, and/or frequency of monitoring (e.g. via house calls) a given patient while providing drug therapy to slow the progression, and increase attention to prevent adverse events such as falls, and/or when to transit patients to or from medication or dosage a to b or c or none, e.g. certain drug therapies, e.g. to slow progression, say of dementia in a case of early detection of cognitive deterioration e.g. of Alzheimer's and other forms of dementia, prioritization of healthcare resources and/or triage, between or over a population of persons with various cognitive states.

Also, embodiments herein may provide accurate, objective, and inexpensive electronic clinical documentation of cognitive status which may then be uploaded in the context of electronic claims submitted to Medicare's or Medicaids' electronic claims processing system by health provider systems, to support the necessity of services billed by the health provider, and to facilitate, simplify, and enhance accuracy of computerized assessment and documentation processes (relative e.g. to conventional assessment protocols that traditionally include periodic (once a week/month/year) questionnaires/patient-reported outcomes and physical tests like TUG, STS, and Rhomberg Balance.

It is appreciated that embodiments provide significant advantages, even relative to cognitive assessment tools which can be administered by paramedics within five minutes or less, e.g. because embodiments herein provide continuous rather than haphazard occasional assessment, and/or because embodiments herein are more automated, hence are more standard. At least for these reasons, more accurate and/or earlier decision support results.

Embodiments herein leverage continuous mobility tracking, to yield cognitive status indications which are standard and cost-effective. The cognitive status indications are also more accurate, relative to conventional indications, due to their being derived from continuous tracking of cognitive status, in contrast to conventional cognitive status indications which are not derived from continuous tracking of cognitive status. These superior cognitive status indications may be used as inputs to drive a wide variety of applications, such as but not limited to early detection of dementia, evaluation of the efficacy of medication and treatment (which cannot be assumed to remain stable over time), and facilitating allocation of caregiver and service provider time including, e.g., appointments-management, based on patients' real-life or up-to-date status, rather than on occasional, hence haphazard, or outdated evaluations thereof.

It is appreciated that the system herein facilitates efficient reduction of collateral damage which, only too often, results from seniors or other end-users who are already experiencing cognitive status deterioration, but the health system has not yet detected this, hence has not yet provided solutions to the end-user's new and reduced circumstances, e.g. because the senior is between appointments with his health provider. For example, the system herein motivates insurance providers to give their end-users a discount if they consent to continuous tracking of cognitive status according to embodiments herein, because the continuous tracking enables end-users to be more closely watched, hence supported, which in turn reduces injury and harm to these end-users, which is both beneficial to them and cost-effective enough to the insurance providing organization, to motivate the organization to, in turn, provide financial motivation to its end-users to onboard the system herein.

It is appreciated that the system herein facilitates efficient reduction of collateral damage which, only too often, results from accidents, e.g. resulting from driving under the influence of alcohol or other substances. A law enforcement organization may incentivize individuals to do less harm to themselves and/or others, if the penalty or sanction against individuals caught driving ‘under the influence’ included a requirement, perhaps with other options which a user may select instead, to consent to continuous tracking of cognitive status based on monitoring of an individual phone to enable future periods of substance abuse while driving to be identified; it is appreciated that systems which identify whether or not a person is currently driving are known. For example, U.S. patent Ser. No. 11/889,015 entitled “System and methods to facilitate safe driving”, the entire disclosure of which is hereby incorporated herein by reference, describes a vehicle safety system operating on a driver's cellphone and comprising selective blocking apparatus, controlled by a processor, for blocking specified functionalities of the cellphone, only when the cellphone is in a moving vehicle, and also describes a process to identify the presence of a cellphone in a driver's seat area by receiving and analyzing RF signals of the cellphone's modem—in order to take certain action only vis a vis a driver's cellphone, and not vis a vis other (passenger) cellphones present in a moving vehicle.

Another advantage of the more continuous monitoring, which embodiments herein provide, relative to conventional monitoring which is far more occasional, is for improving care for those who tend to experience swings or fluctuations in their cognitive status, such that their cognitive deficits fluctuate in severity over time. Not a few persons suffer from such fluctuations, such as but not limited to substance abusers, as well as individuals with schizophrenia or bipolar disorder, who may flit between episodes of low cognitive status (e.g. manic and/or depressive episodes) and periods of relative stability. It is believed that the cognitive status of individuals with bipolar disorder and schizophrenia can change even within hours, e.g. during intense mood episodes, where, during a manic episode, a bipolar person might exhibit rapid, disorganized thinking, whereas during a depressive episode, the same person may experience significant cognitive slowing and/or difficulty concentrating. Also, for a person with schizophrenia, it is believed that cognitive status changes within hours are possible, e.g. during severe episodes of psychosis or agitation in which cognitive status can rapidly become significantly impaired e.g. in terms of attention and/or memory, and/or executive function. it is appreciated that continuous monitoring may allow these rapid changes to be detected much more quickly than conventional protocols for monitoring such person support, and, responsively, rapid changes in the affected person's medication, say, may be made to positively influence the person's cognitive status.

Another advantage of embodiments herein is that due to ease of use, the number of users undergoing cognitive testing via embodiments herein may be much greater than is currently the case, given that only labor-intensive cognitive testing tools are conventionally employed. As a result of the smaller number of users now undergoing cognitive testing, the testing is not suited to small neurodiverse populations, such as persons with relatively rare intellectual and/or developmental disabilities, such as people with Down's syndrome, even though these are at an increased risk of developing Alzheimer's. This is because testing is typically hard and costly to adequately assess or adapt to small populations, unless big data is available, in which case development of norms for even small sub-populations becomes possible.

Another advantage of embodiments herein is that, in practice, only some persons (e.g. fewer than one-third of Medicare beneficiaries, according to some estimates) in fact receive a conventional formal cognitive assessment at annual wellness visits. A faster, more efficient cognitive status testing method, e.g. as described herein, is expected to improve this phenomenon.

Another advantage of embodiments herein is collection of large amounts of gait data over weeks, months, and even years, to show long-term trends of cognitive status/cognitive decline.

Another advantage of embodiments herein is the facilitation of early cognitive impairment screening to assess cognitive performance and decline (e.g. to screen for cognitive decline associated with dementia and its subtypes, such as Alzheimer's disease, as well as other neurodegenerative diseases, including Parkinson's disease (PD), Multiple Sclerosis (MS), and Creutzfeldt-Jakob Disease (CJD), given that two simultaneously performed tasks may interfere and compete for brain cortical resources.

References to fusion herein are typically designed to use extracted environmental conditions to add information to semantic segmentation, e.g. as described herein with reference to layer A, of motion data e.g. by identifying when motion measurements (IMU/gait analysis) indicate that the user completed a given passive walk under certain environmental conditions e.g. in a crowded area, or outdoors, or on an incline, or on a rough vs. smooth surface. Thus motion data may be combined with extracted environmental conditions to enable the system to compare only mobility measures collected in similar contexts e.g. to compare mobility measures captured while walking on a level, smooth surface in fine weather, without congestion but with dual tasking, to mobility measures captured under the same “regular” conditions but without dual tasking. This enables the effect of dual task to be isolated when conducting passive walk measurement.

Herein, “passive” is intended to include IMU data collection from a user's phone's IMU, which does not require user cooperation each time the IMU data is collected and in fact the user may be unaware that IMU data is being collected e.g. because IMU data collection and subsequent analysis do not require that the user be prompted to provide any information or to perform any particular action. For example, the system typically does not require a user to be prompted to walk or to indicate whether or not s/he is walking. Instead, the disclosures of co-owned U.S. Ser. No. 18/975,890 entitled “Advanced Pedestrian Navigation Based on Inertial Gait Analysis and GPS Data” and/or of co-owned US20220111257 entitled “System . . . for Sensor-Based Enhancement of Physical Rehabilitation” (both of which are hereby incorporated herein by reference in their entirety) may be used to automatically classify a given time-interval as including gait or not e.g. by determining whether the user is ambulating during that interval. Then, IMU data may if desired to be collected only during such intervals which, as automatically determined, do include ambulation as opposed to intervals in which an end-user is stationary e.g.

It is appreciated that any suitable output indication generating functionality may be employed to generate an output indication of any result or prediction or parameter described herein, which may be yielded by any embodiment of the system herein; the output indication may be provided to any suitable entity in any suitable manner (e.g. automatic SMS to HMO and/or a report document which may be consumed via web dashboard or via API with EMR software platform such as Epic Systems or Cerner, or via email to an email address, stored in the system, of the entity).

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required. or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof may also be provided as methods and operations therewithin, and functionalities described or illustrated as methods and operations therewithin may also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via WiFi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein, which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. A health monitoring method comprising:
using a cellphone having an IMU, to measure:
at least one gait parameter of a human bearing the cellphone during a first period in which the human is known to be dual-tasking; and
at least one gait parameter of the human bearing the cellphone during a second period in which the human is known not to be dual-tasking; and
using a hardware processor to receive the gait parameters and to compute at least one difference between the gait parameters during the first and second periods, and, accordingly, to generate a cognitive health alert.

2. A method according to claim 1 and wherein the health alert comprises a cognitive status indication provided by automatically and repeatedly, e.g. according to a programmed schedule or periodically, thresholding said at least one difference, to provide close tracking of cognitive status which results from repeatedly receiving up-to-date gait parameters and repeatedly using the hardware processor to compute said difference.

3. A method according to claim 1 and wherein the cognitive status indication comprises an output indication sensible by humans which may be generated at a location remote from the cellphone.

4. A method according to claim 1 and wherein the cognitive status indication comprises an output signal fed to an external computer system, which receives and conducts comparisons between said output signal and between similar output signals from a multiplicity of persons bearing cellphones and, responsively, continuously re-ranks persons to prioritize persons according to said output signals provided by said method and by said persons' cellphones.

5. A method according to claim 1 and wherein said output indication is generated each time the difference falls below at least one baseline range of gait parameter values.

6. A method according to claim 1 and wherein said hardware processor used to compute the at least one difference, comprises the cellphone's hardware processor.

7. A method according to claim 1 and wherein at least one of the first and second periods is determined automatically by determining whether a human is or is not dual-tasking during at least one time-period, e.g. constantly, to enable cognitive monitoring, even without any active cooperation from the user.

8. A method according to claim 1 and wherein a single cognitive status indication is used to generate plural output signals fed to plural external computer systems respectively, each of which is tasked with multifactorial data processing tasks respectively, thereby to define plural multifactorial data processing tasks, all of which depend inter alia on cognitive status.

9. A health monitoring system comprising:
a hardware processor interfacing with a cellphone's IMU, to collect:
at least one gait parameter of a human bearing the cellphone during a first period in which the human is known to be dual-tasking; and
at least one gait parameter of the human bearing the cellphone during a second period in which the human is known not to be dual-tasking;
and wherein the hardware processor is configured for computing at least one difference between the gait parameters during the first and second periods, and, accordingly, commanding the cellphone to generate a cognitive health alert which is typically sensible by a caregiver.

10. A method according to claim 1 and wherein the gait parameters are measured passively without user cooperation, thereby to enable the cognitive health alert to be generated without enlisting user cooperation.

11. A method according to claim 10 wherein the first and second periods are selected to be comparable.

12. A computer program product, comprising a non-transitory tangible computer readable medium having computer readable program code embodied therein, said computer readable program code adapted to be executed to implement a health monitoring method comprising:
using a cellphone having an IMU, to measure:
at least one gait parameter of a human bearing the cellphone during a first period in which the human is known to be dual-tasking; and
at least one gait parameter of the human bearing the cellphone during a second period in which the human is known not to be dual-tasking; and
using a hardware processor to receive the gait parameters and to compute at least one difference between the gait parameters during the first and second periods, and, accordingly, to generate a cognitive health alert.
